(19) United States
(12) Patent Application Publication
(10) Pub. No.: US 2005/0215582 A1
(43) Pub. Date: Sep. 29, 2005

(54) Substituted pyrrolopyridines

(76) Inventors: Peter Aadal Nielsen, Lund (SE); Thomas Brimert, Lund (SE); Anna Kristoffersson, Lund (SE); Tero Linnanen, Lund (SE); Peter Sjo, Lund (SE)

Correspondence Address: FISH & RICHARDSON P.C., 225 FRANKLIN STREET, BOSTON, MA, 02110, US

(21) Appl. No.: 10/524,361

(22) PCT Filed: Aug. 13, 2003
(86) PCT No.: PCT/SE03/01275

§ 371 Date: Feb. 10, 2005

(30) Foreign Application Priority Data
Aug. 14, 2002 (SE) 0202463-6

(51) Int. Cl. A61K 31/4745; C07D 471/02

(52) U.S. Cl. 514/300; 546/113

(57) ABSTRACT

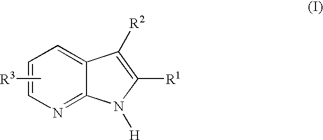

There are provided novel compounds of formula (I) wherein R¹, R² and R³ are as defined in the specification and pharmaceutically acceptable salts thereof; together with processes for their preparation, compositions containing them and their use in therapy. The compounds are inhibitors of the kinase Itk.

### FIELD OF THE INVENTION

This invention relates to novel 2-heteroaryl- and 2-aryl-7-azaindole[2-(hetero)aryl-1H-pyrrolo[2,3-b]pyridine]derivatives, processes for their preparation, intermediates thereto, pharmaceutical compositions comprising them, and their use in therapy.

### BACKGROUND OF THE INVENTION

Inducible T cell Kinase (Itk) is a member of the Tec-family of cytosolic protein tyrosine kinases. In mammalians, this family also includes Btk, Tec, Bmx, and Txk. These kinases regulate various immune cell functions that integrate signals given by the other cytosolic tyrosine kinases as well as serine/threonine kinases, lipid kinases, and small G proteins. Tec-family kinases have the following general structure: a N-terminal pleckstrin-homology (PH) domain, a Tec-homology domain that includes a Btk motif and one or two proline-rich (PR) motifs, a SH3 domain, a SH2 domain and a c-terminal catalytic (SH1) domain. These kinases are expressed exclusively in hematopoietic tissues, with the exception of Tec and Bmx that have also been detected in endothelial cells. The cellular distribution is different for the Tec-family members. For example, Itk is expressed by T cells, NK cells and mast cells, whereas Btk is expressed by all hematopoietic cells except T cells. Thus, hematopoietic cells may express one or several Tec-family kinases. For example, T cells express Itk, Tec and Txk, and mast cells express Btk, Itk and Tec.

Btk is by far the most extensively studied among the Tec-family kinases, due to its association with X-linked agammaglobulinemia (XLA), and Btk is currently the only Tec-family kinase with a known human phenotype. XLA patients are virtually devoid of mature B cells and their Ig levels are strongly reduced.

Itk^{-/-} mice show defects in T cell activation and differentiation. T helper 2 (Th2) differentiation is disrupted in these mice, whereas Th1 differentiation is apparently intact.

In T and B cells, signalling through T cell receptors and B cell receptors leads to activation of Itk and Btk, respectively. Downstream of Itk and Btk a number of different messengers are engaged; scaffolding proteins (SLP-76, LAT, SLP-65), Src kinases, MAP kinases, and PI3-K. These events are followed by PLC-γ activation that leads to IP3 generation and sustained Ca²⁺ flux, and subsequently activation of transcription factors. PLC-γ1 has been suggested as a direct substrate for Itk.

In T cells, Itk (and Tec) may also mediate signalling through the CD28 co-receptor. Furthermore, Itk has in T cells been implicated in the activation of β-integrin.

Signalling from Tec-family kinases can also be regulated by PH domain-mediated plasma membrane localization, and by Src-family-mediated phosphorylation of critical tyrosine residues. Interestingly, Itk, Btk and Txk have recently been shown to translocate to the nucleus after activation.

From studies using Itk−/− mice, it has been proposed that Itk is required for Th2 but not Th1 cell development. This was demonstrated in the N. brasiliensis and L. major infection models where the Itk−/− animals are protected in the Leishmania model indicating an intact Th1 response, whereas they are susceptible to infection with N. Brasiliensis that requires an intact Th2 response for resolution of the infection. This indicates that modulation of Itk activity may prove useful for treatment of Th2-driven disorders and conditions.

We have identified the critical role of Itk in regulating important mast cell and basophil functions and established that the activity of mast cells or basophils may be inhibited through inhibition of Itk. Thus Itk inhibitors may be used as pharmaceutical agents for the treatment of mast cell-driven or basophil-driven conditions or diseases. In particular, we have identified Itk as a target for inhibiting several key events in both acute and late phase allergic reactions common to allergic rhinitis and asthma.

WO 01/47922 discloses substituted aza- and diaza-indoles as kinase inhibitors, in particular, as inhibitors of the protein tryosine kinase Syk. The compounds disclosed in WO 01/47922 are not within the generic scope of the present application.

The present invention discloses novel substituted 2-heteroaryl- and 2-aryl-7-azaindoles that have activity as Itk inhibitors and are thereby useful as pharmaceuticals, particularly for the treatment of allergic rhinitis and of asthma.

### DISCLOSURE OF THE INVENTION

The present invention provides a compound of formula (I): 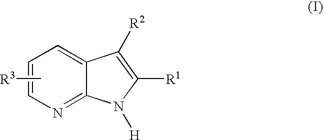
wherein:
R¹ represents phenyl or a five or six membered aromatic heterocyclic ring containing 1 to 3 heteroatoms selected independently from O, S and N; said phenyl or aromatic heterocyclic ring being optionally substituted by one or more substituents selected independently from halogen, C1 to 4 alkyl, C1 to 4 alkoxy, CO₂R⁴ or a group -K-L-M;
K represents O, NR¹² or a bond;
L represents C1 to 4 alkyl optionally further substituted by OH or OMe; or L represents a bond;
M represents NR¹³R¹⁴or OR¹⁵;
R¹³ and R¹⁴ independently represent H or C1 to 4 alkyl; or the group —NR¹³R¹⁴ together represents a saturated 5 to 7 membered azacyclic ring optionally incorporating one further heteroatom selected from O, S and NR¹⁶;
R¹⁶ represents H, C1 to 4 alkyl or C2 to 4 alkanoyl;
R² represents a saturated or partially unsaturated 3 to 7 membered ring, optionally including 1 or 2 heteroatoms independently selected from O, N and S(O)ₙ and optionally incorporating 1 or 2 carbonyl groups; and optionally substituted by halogen, OH, C1 to 4 alkyl, C1 to 4 alkoxy, CHO, C2 to 4 alkanoyl, C1 to 4 alkylsulphonyl, CO₂R⁵, C(Z)NR¹⁷R¹⁸ or pyrrolidine-2,5-dione; said C1 to 4 alkylsulphonyl group being optionally further substituted by 1H-isoindole-1,3(2H)-dione;
Z represents O or S;
R¹⁷ and R¹⁸ independently represent H or C1 to 4 alkyl; or the group —NR¹⁷R¹⁸ together represents a saturated 5 to 7 membered azacyclic ring optionally incorporating one further heteroatom selected from O, S and NR¹⁹;
R³ represents H, halogen, C1 to 4 alkyl, C1 to 4 alkoxy or cyano;
R⁴, R⁵, R¹², R¹⁵ and R¹⁹ independently represent H or C1 to 4 alkyl;
n represents an integer 0, 1 or 2;
and pharmaceutically acceptable salts thereof.

The compounds of formula (I) may exist in enantiomeric forms. All enantiomers, diastereoisomers, racemates and mixtures thereof are included within the scope of the invention.

Compounds of formula (I) may also exist in various tautomeric forms. All possible tautomeric forms and mixtures thereof are included within the scope of the invention.

Unless otherwise indicated, the term “C1 to 4 alkyl” referred to herein denotes a straight or branched chain alkyl group having from 1 to 4 carbon atoms. Examples of such groups include methyl, ethyl, n-propyl, i-propyl, n-butyl, i-butyl and t-butyl. The term “C1 to 2 alkyl” is to be interpreted analogously.

Unless otherwise indicated, the term “C1 to 4 alkoxyl” referred to herein denotes an oxygen substituent bonded to a straight or branched chain alkyl group having from 1 to 4 carbon atoms. Examples of such groups include methoxy, ethoxy, n-propoxy, n-butoxy, i-butoxy and s-butoxy. The term “C1 to 2 alkoxy” is to be interpreted analogously.

Unless otherwise indicated, the term “C2 to 4 alkanoyl” referred to herein denotes a carbonyl group attached to a straight or branched chain alkyl group having from 1 to 3 carbon atoms. Examples of such groups include acetyl and propionyl.

Unless otherwise indicated, the term “C1 to 4 alkylsulphonyl” referred to herein denotes a sulphonyl group, —SO₂—, attached to a straight or branched chain alkyl group having from 1 to 4 carbon atoms. Examples of such groups include methylsulphonyl, ethylsulphonyl and isopropylsulphonyl.

Unless otherwise indicated, the term “halogen” referred to herein denotes fluorine, chlorine, bromine and iodine.

Examples of a five or six membered aromatic heterocyclic ring containing 1 to 3 heteroaloms independently selected independently from O, S and N include furan, thiophene, pyrrole, pyridine, imidazole, thiazole, oxazole, isoxazole, isothiazole, triazole, oxadiazole, pyrazine and pyrimidine.

Examples of a saturated or partially unsaturated 3 to 7 membered ring, optionally including 1 or 2 heteroatoms independently selected from O, N and S(O)ₙ and optionally incorporating 1 or 2 carbonyl groups include cyclopropane, cyclopentane, cyclohexane, cycloheptane, pyrrolidine, 1-piperidine, 2-piperidine, 3-piperidine, 4-piperidine, morpholine, thiomorpholine, piperazine, pyrrolidinone, oxazolidinone, piperidinone, tetrahydrofuran, cyclopentene, cyclohexene, dihydroimidazole and dehydropiperidine.

Examples of a saturated 5 to 7 membered azacyclic ring optionally incorporating one further heteroatom selected from O, S and NR include pyrrolidine, piperidine, morpholine and piperazine.

In one embodiment, R¹ in formula (I) represents optionally substituted phenyl, furyl, thienyl, thiazolyl, pyrrolyl or oxazolyl. In another embodiment, R¹ represents optionally substituted phenyl, furyl or pyrrolyl.

In one embodiment, R¹ in formula (I) represents phenyl optionally substitutedby halogen, C1 to 4 alkyl or a group -K-L-M.

In one embodiment, K represents O. In another embodiment, K represents NR¹².

In one embodiment, L represents C1 to 4 alkyl.

In one embodiment, M represents NR¹³R¹⁴.

In one embodiment, the group -K-L-M represents —O—(CH₂)₃—NR¹³R¹⁴. In another embodiment, the group -K-L-M represents —O—(CH₂)₃—N(CH₃)₂.

In one embodiment, R³ in formula (I) is at the 5-position of the azaindole ring system. In one embodiment, R³ represents halogen, methyl or cyano. In another embodiment, R³ represents bromo. In another embodiment, R³ represents chloro. In another embodiment, R³ represents methyl.

In one embodiment, R² in formula (I) represents a saturated or partially unsaturated 3 to 7 membered ring, optionally including 1 or 2 heteroatoms independently selected from O, N and S(O)ₙ; and optionally substituted by OH, C1 to 4 alkyl, C2 to 4 alkanoyl, C1 to 4 alkylsulphonyl or C(Z)NR¹⁷R¹⁸.

In one embodiment, R² represents optionally substituted piperazine.

In one embodiment, R² represents optionally substituted 3-piperazine. In another embodiment, R² represents optionally substituted 4-piperazine. In another embodiment, R² represents cyclopropane or cyclohexene.

In one embodiment, the present invention provides a compound of formula (I) wherein R¹ represents phenyl or a five or six membered aromatic heterocyclic ring containing 1 to 3 heteroatoms selected independently from O, S and N; said phenyl or aromatic heterocyclic ring being optionally substituted by one or more substituents selected independently from halogen, C1 to 4 alkyl, C1 to 4 alkoxy or CO₂R⁴; R² represents a saturated or partially unsaturated 4 to 7 membered ring, optionally including 1 or 2 heteroatoms independently selected from O, N and S(O)ₙ and optionally incorporating 1 or 2 carbonyl groups; and optionally substituted by halogen, OH, C1 to 4 alkyl, C1 to 4 alkoxy, C1 to 4 alkanoyl, C1 to 4 alkylsulphonyl or CO₂R⁵; R³ represents H, halogen, C1 to 4 alkyl, C1 to 4 alkoxy or cyano; R⁴ and R⁵ independently represent H or C1 to 4 alkyl; n represents an integer 0, 1 or 2; and pharmaceutically acceptable salts thereof;

Particular compounds according to the present invention include:
{3-[4-(5-chloro-3-cyclopropyl-1H-pyrrolo[2,3-b]pyridin-2-yl)phenoxy]propyl}dimethylamine;
{3-[4-(5-chloro-3-cyclohex-1-en-1-yl-1H-pyrrolo[2,3-b]pyridin-2-yl)phenoxy]propyl}dimethylamine;
tert-butyl 3-(2-{4-[3-(dimethylamino)propoxy]phenyl}-5-methyl-1H-pyrrolo[2,3-b]pyridin-3-yl)piperidine-1-carboxylate;
2-(2-furyl)-5-methyl-3-piperidin-3-yl-1H-pyrrolo[2,3-b]pyridine;
3-[2-(2-furyl)-5-methyl-1H-pyrrolo[2,3-b]pyridin-3-yl]piperidine-1-carboxamide;
5-chloro-3-piperidin-4-yl-2-(1H-pyrrol-3-yl)-1H-pyrrolo[2,3-b]pyridine;
tert-butyl 4-(5-chloro-2-{4-[3-(dimethylamino)propoxy]phenyl}-1H-pyrrolo[2,3-b]pyridin-3-yl)piperidine-1-carboxylate;
{3-[4-(5-chloro-3-piperidin-4-yl-1H-pyrrolo[2,3-b]pyridin-2-yl)phenoxy]propyl}dimethylamine;
[3-(4-{5-chloro-3-[1-(methylsulfonyl)piperidin-4-yl]-1H-pyrrolo[2,3-b]pyridin-2-yl}phenoxy)propyl]dimethylamine;
4-(5-chloro-2-{4-[3-(dimethylamino)propoxy]phenyl}-1H-pyrrolo[2,3-b]pyridin-3-yl)piperidine-1-carbaldehyde;
4-(5-chloro-2-{4-[3-(dimethylamino)propoxy]phenyl}-1H-pyrrolo[2,3-b]pyridin-3-yl)piperidine-1-carboxamide;
3-(2-{4-[3-(dimethylamino)propoxy]phenyl}-5-methyl-1H-pyrrolo[2,3-b]pyridin-3-yl)-N,N-dimethylpiperidine-1-carboxamide;
3-(2-{4-[3-(dimethylamino)propoxy]phenyl}-5-methyl-1H-pyrrolo[2,3-b]pyridin-3-yl)-N-isopropylpiperidine-1-carboxamide;
dimethyl[3-(4-{5-methyl-3-[1-(pyrrolidin-1-ylcarbonyl)piperidin-3-yl]-1H-pyrrolo[2,3-b]pyridin-2-yl}phenoxy)propyl]amine;
[3-(4-{3-[1-(isopropylsulfonyl)piperidin-3-yl]-5-methyl-1H-pyrrolo[2,3-b]pyridin-2yl}phenoxy)propyl]dimethylamine;
(3-{4-[3-(1-acetylpiperidin-3-yl)-5-methyl-1H-pyrrolo[2,3-b]pyridin-2-yl]phenoxy}propyl)dimethylamine;
3-(2-{4-[3-(dimethylamino)propoxy]phenyl}-5-methyl-1H-pyrrolo[2,3-b]pyridin-3-yl)-N-methylpiperidine-1-carbothioamide;
2-(2-{[3-(2-{4-[3-(dimethylamino)propoxy]phenyl}-5-methyl-1H-pyrrolo[2,3-b]pyridin-3-yl)piperidin-1-yl]sulfonyl}ethyl)-1H-isoindole-1,3(2H)-dione;
3-[3-(2-{4-[3-(dimethylamino)propoxy]phenyl}-5-methyl-1H-pyrrolo[2,3-b]pyridin-3-yl]piperidin-1-yl)pyrrolidine-2,5-dione;
dimethyl[3-(4-{5-methyl-3-[1-(methylsulfonyl)piperidin-3-yl]-1H-pyrrolo[2,3-b]pyridin-2-yl}phenoxy)propyl]amine;
5-bromo-2-(4-methoxy-phenyl)-3-piperazin-1-yl-1H-pyrrolo[2,3-b]pyridine;
5-bromo-2-(4-methoxyphenyl)-3-(4-methylpiperazin-1-yl)-1H-pyrrolo[2,3-b]pyridine;
4-[5-bromo-2-(4-methoxy-phenyl)-1H-pyrrolo[2,3-b]pyridin-3-yl]-piperazine-1-carboxylic acid tert-butyl ester;
5-bromo-2-phenyl-3-morpholin-4-yl-1H-pyrrolo[2,3-b]pyridine;
5-bromo-3-(4-methanesulfonylpiperazin-1-yl)-2-(4-methoxy-phenyl)-1H-pyrrolo[2,3-b]pyridine;
4-[5-bromo-2-(4-methoxy-phenyl)-1H-pyrrolo[2,3-b]pyridin-3-yl]-piperazine-1-carbaldehyde;
and pharmaceutically acceptable salts thereof.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

In a further aspect the invention provides a process for the preparation of a compound of formula (I) which comprises:
a) reaction of a compound of formula (II): 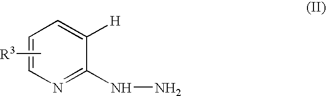
   in which R³ is as defined in formula (I), with a compound of formula (III): 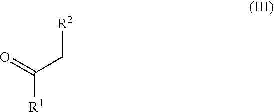
   in which R¹ and R² are as defined in formula (I); or
b) arylation of a compound of formula (IV) 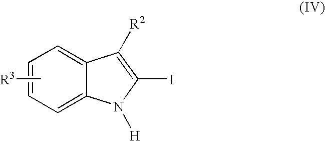
   wherein R² and R³ are as defined above, with a boronic acid of formula R¹—B(OH)₂ wherein R¹ is is defined above;
   and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.

Process (a) may be carried out by heating together at a suitable temperature and preferably in an inert atmosphere the compounds of formulae (II) and (III), optionally in the presence of an inert solvent. Preferably the reaction is carried out at a temperature between 100° C. and 250° C., preferably in the absence of a solvent. Suitable reaction times are generally from 5 minutes to 3 hours.

Alternatively process (a) may be carried out in two steps. In the first step, the compounds of formulae (II) and (IlI) are condensed together to give an intermediate hydrazone of formula (V) 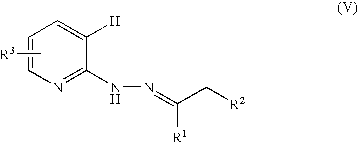
wherein R¹, R² and R³ are as defined in formula (I).

And in a second step the hydrazone (V) is cyclised by heating under similar conditions to those used for the single step process above. The condensation of compounds of formulae (II) and (III) to give the hydrazone (V) is generally carried out in an inert solvent such as benzene or toluene in the presence of an acid catalyst such as acetic acid or p-toluenesulphonic acid with removal of water by azeotropic distillation.

In process (b), the arylation may be performed in the presence of a suitable palladium catalyst using well known cross-coupling conditions such as those described by A. Suzuki, J. Organomet. Chem. 1999, 576, 147-168.

2-Iodo azaindoles of formula (IV) may be prepared, for example, according to the following Scheme:

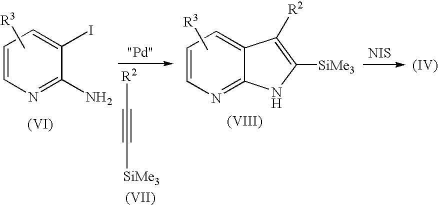

For the cyclization step, conditions as described by F. Ujjainwalla and D. Warner, Tetrahedron Letters, 1998, 39, 5355-5358 may be used. The silyl-iodo-exchange can be performed using N-iodosuccinimide (NIS) according to the protocol described by S. Berteina Raboin et al., Org. Letters, 2002, 4, 2613-261. Compounds of formula (VI) may, for example, be obtained by iodination of suitably substituted 2-amino-pyridines using the conditions described by G. A. Olah et al., J. Org. Chem., 1993, 58, 3194-3195.

Aryl boronic acids R¹—B(OH)₂ are either commercially available or may be prepared using well known literature procedures, such as from the corresponding aryl halides.

Compounds of formula (I) in which R¹ represents an aromatic ring substituted by a group -K-L-M may, when K represents O, be prepared by alkylation of the corresponding compound wherein the aromatic ring is substituted by OH, using reactions that will be readily apparent to the man skilled in the art. Compounds of formula (I) in which R¹ represents an aromatic ring substituted by a group -K-L-M may, when K represents NR¹², be prepared by reductive amination of the corresponding compound wherein the aromatic ring is substituted by NHR¹², using reactions that will be readily apparent to the man skilled in the art.

Alkynes (VII) may be synthezised starting from a suitably protected aldehyde by analogy to the protocol described by K. Miwa, T. Aoyama and T. Shioiri, Synlett., 1994, 107-108.

Salts of compounds of formula (I) may be formed by reacting the free base or a salt, enantiomer, tautomer or protected derivative thereof, with one or more equivalents of the appropriate acid. The reaction may be carried out in a solvent or medium in which the salt is insoluble, or in a solvent in which the salt is soluble followed by subsequent removal of the solvent in vacuo or by freeze drying. Suitable solvents include, for example, water, dioxan, ethanol, 2-propanol, tetrahydrofuran or diethyl ether, or mixtures thereof. The reaction may be a metathetical process or it may be carried out on an ion exchange resin.

Compounds of formula (I) and intermediate compounds thereto may be prepared as such or in protected form. The protection and deprotection of functional groups is, for example, described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

The compounds of the invention and intermediates may be isolated from their reaction mixtures, and if necessary further purified, by using standard techniques.

The compounds of formula (I) may exist in enantiomeric or diastereoisomeric forms or mixtures thereof, all of which are included within the scope of the invention. The various optical isomers may be isolated by separation of a racemic mixture of the compounds using conventional techniques, for example, fractional crystallisation or HPLC. Alternatively, the individual enantiomers may be made by reaction of the appropriate optically active starting materials under reaction conditions that will not cause racemisation.

Intermediate compounds may also exist in enantiomeric forms and may be used as purified enantiomers, diastereomers, racemates or mixtures thereof.

According to a further aspect of the invention we provide a compound of formula (I) or a pharmaceutically acceptable salts thereof, for use as a medicament.

The compounds of formula (I), and their pharmaceutically acceptable salts are useful because they possess pharmacological activity in animals. The compounds of formula (I) have activity as pharmaceuticals, in particular as modulators of kinase activity, especially Itk kinase activity, and as such are predicted to be useful in therapy. They may be used in the treatment or prophylaxis of allergic, autoimmune, inflammatory, proliferative and hyperproliferative diseases and immune-mediated diseases including rejection of transplanted organs or tissues and Acquired Immunodeficiency Syndrome (AIDS).

Thus, another aspect of the invention provides the use of a compound of fomnula (I) or a pharmaceutically acceptable salt thereof, in the manufacture of a medicament for the treatment or prophylaxis of diseases or conditions in which inhibition of Itk activity is beneficial; and a method of treating, or reducing the risk of, diseases or conditions in which inhibition of Itk activity is beneficial which comprises administering to a person suffering from or at risk of, said disease or condition, a therapeutically effective amount of a compound of formula (I) or a pharmaceutically acceptable salt thereof.

Examples of these conditions are:

(1) (the respiratory tract) airways diseases including chronic obstructive pulmonary disease (COPD) such as irreversible COPD; asthma, such as bronchial, allergic, intrinsic, extrinsic and dust asthma, particularly chronic or inveterate asthma (for example, late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; inembranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia; sinusitis, chronic rhinosinusitis, nasosinusal polyposis; pulmonary fibrosis;

(2) (bone and joints) rheumatoid arthritis, seronegative spondyloarthropathies (including ankylosing spondylitis, psoriatic arthritis and Reiter's disease), Behcet's disease, Sjogren's syndrome and systemic sclerosis;

(3) (skin) psoriasis, atopical dermatitis, contact dermatitis and other eczmatous dermitides, seborhoetic dermatitis, Lichen planus, Pemphigus, bullous Pemphigus, Epiderrnolysis bullosa, urticaria, angiodermas, vasculitides, erythemas, cutaneous eosinophilias, uveitis, Alopecia areata and vernal conjunctivitis;

(4) (gastrointestinal tract) Coeliac disease, proctitis, eosinopilic gastro-enteritis, mastocytosis, Crohn's disease, ulcerative colitis, food-related allergies which have effects remote from the gut, for example, migaine, rhinitis and eczema;

(5) (other tissues and systemic disease) multiple sclerosis, atherosclerosis, Acquired Immunodeficiency Syndrome (AIDS), lupus erythematosus, systemic lupus, erythematosus, Hashimoto's thyroiditis, myasthenia gravis, type I diabetes, nephrotic syndrome, eosinophilia fascitis, hyper IgE syndrome, lepromatous leprosy, sezary syndrome and idiopathic thrombocytopenia pupura; tuberculosis;

(6) (allograft rejection) acute and chronic following, for example, transplantation of kidney, heart, liver, lung, bone marrow, skin and cornea; and chronic graft versus host disease.

We are particularly interested in Th2-driven and/or mast cell-driven and/or basophil-driven conditions or diseases.

Thus, a more particular aspect of the invention provides the use of a compound of formula (I) or a pharmaceutically acceptable salt thereof, in the manufacture of a medicament for the treatment or prophylaxis of Th2-driven and/or mast cell-driven and/or basophil driven diseases or conditions; and a method of treating, or reducing the risk of, Th2-driven and/or mast cell-driven and/or basophil driven diseases or conditions which comprises administering to a person suffering from or at risk of, said disease or condition, a therapeutically effective amount of a compound of formula (I) or a pharmaceutically acceptable salt thereof.

In a preferred aspect of the invention, we provide a method for the treatment or prevention of a reversible obstructive airway disease, especially asthma, which comprises administering a therapeutically effective amount of a compound of formula (I) or a pharmaceutically acceptable salt thereof to a human that is suffering from or susceptible to the disease. We also provide the use of a compound of formula (I) or a pharmaceutically acceptable salt thereof in the manufacture of a medicament for the treatment or prevention of a reversible obstructive airway disease, especially asthma.

In another preferred aspect of the invention, we provide a method for the treatment or prevention of rhinitis which comprises administering a therapeutically effective amount of a compound of formula (I) or a pharmaceutically acceptable salt thereof to a human that is suffering from or susceptible to rhinitis, especially allergic rhinitis. We also provide the use of a compound of formula (I) or a pharrnaceutically acceptable salt thereof in the manufacture of a medicament for the treatment or prevention of rhinitis, especially allergic rhinitis.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dose of thecompound to be administered will depend on the compound employed, the disease being treated, the mode of administration, the age, weight and sex of the patient. Such factors may be determined by the attending physician. However, in general, satisfactory results are obtained when the, compounds are administered to a human at a daily dosage of between 0.1 mg/kg to 100 mg/kg (measured as the active ingredient).

The compounds of formula (I) may be used on their own, or in the form of appropriate pharmaceutical formulations comprising the compound of the invention in combination with a pharmaceutically acceptable diluent, adjuvant or carrier. Particularly preferred are compositions not containing material capable of causing an adverse reaction, for example, an allergic reaction. Conventional procedures for the selection and preparation of suitable pharmaceutical formulations are described in, for example, “Pharmaceuticals—The Science of Dosage Form Designs”, M. E. Aulton, Churchill Livingstone, 1988.

According to the invention, there is provided a pharmaceutical formulation comprising preferably less than 95% by weight and more preferably less than 50% by weight of a compound of formula (I) in admixture with a pharmaceutically acceptable diluent or carrier.

We also provide a method of preparation of such pharmaceutical formulations that comprises mixing the ingredients.

The compounds may be administered topically, for example, to the lungs and/or the airways, in the form of solutions, suspensions, HFA aerosols or dry powder formulations, for example, formulations in the inhaler device known as the Turbuhaler®; or systemically, for example, by oral administration in the form of tablets, pills, capsules, syrups, powders or granules; or by parenteral administration, for example, in the form of sterile parenteral solutions or suspensions; or by rectal administration, for example, in the form of suppositories.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound with a carrier substance, for example, a mono-, di- or polysaccharide, a sugar alcohol, or another poly. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active compound, with or without a carrier substance, is delivered to the patient.

For oral administration the active compound may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum, titanium dioxide, and the like. Alternatively, the tablet may be coated with a suitable polymer dissolved in a readily volatile organic solvent.

For the preparation of soft gelatine capsules, the compound may be admixed with, for example, a vegetable oil or polyethylene glycol. Hard gelatine capsules may contain granules of the compound using either the above mentioned excipients for tablets. Also liquid or semisolid formulations of the drug may be filled into hard gelatine capsules.

Liquid preparations for oral application may be in the form of syrups or suspensions, for example, solutions containing the compound, the balance being sugar and a mixture of ethanol, water, glycerol and propylene glycol. Optionally such liquid preparations may contain colouring agents, flavouring agents, saccharine and/or carboxymethylcellulose as a thickening agent orotherexcipients known to those skilled in art.

The compounds of the invention may also be administered in conjunction with other compounds used for the treatment of the above conditions.

The following Examples are intended to illustrate, but in no way limit the scope of the invention.

General methods All reactions were performed in dried-glassware in an argon atmosphere at room temperature, unless otherwise noted. All reagents and solvents were used as received. Merck Silica gel 60 (0.040-0.063 mm) was used for preparative silica gel chromatography. A Kromasil KR-100-5-C18 column (250×20 mm, Akzo Nobel) and mixtures of acetonitrile/water at a flow rate of 10 ml/min was used for preparative HPLC. Reactions were monitored at 254 nm by analytical HPLC, using a Kromasil C-18 column (150×4.6 mm) and a gradient (containing 0.1% trifluoroacetic acid) of 5 to 100% of acetonitrile in water at a flow rate of 1 ml/min. Evaporations of solvents were performed under reduced pressure using a rotary evaporator at a maximum temperature of 60° C. Products were dried under reduced pressure at about 40° C.

¹H-NMR spectra were recorded on a Varian Inova 400 MHz or Varian Mercury 300 MHz instrument. The central solvent peak of chloroform-d (δ_{H} 7.27 ppm), dimethylsulfoxide-d₆ (δ_{H} 2.50 ppm) or methanol-d₄ (δ_{H} 3.35 ppm) were used as internal references. Low resolution mass spectra were obtained on a Hewlett Packard 1100 LC-MS system equipped with a APCI ionisation chamber.

### EXAMPLE 1

### {3-[4-(5-Chloro-3-cyclopropyl-1H-pyrrolo[2,3-b]pyridin-2-yl)phenoxy]propyyl}dimethylamine

5-Chloro-2-iodo-3-isopropenyl-1H-pyrrolo[2,3-b]pyridine (55 mg, 0.17 mmol), {4-[3-(dimethylamino)propoxy]phenyl}boronic acid (82 mg, 0.37 mmol), potassium carbonate (0.11 g, 0.78 mmol) and 1,1′-bis(diphenylphosphino)ferrocenedichloro-palladium(II) (11 mg, 0.0013 mmol) were suspended in dioxane (10 ml). The mixture was degassed with argon and stirred at 100° C. for 14 h. The reaction mixture was diluted with methanol and stirred with an excess of acidic Dowex 50-W2 ion-exchange resin for 15 min. The resin was washed with methanol and then with methanol containing 10% of aqueous concentrated ammonia. The basic methanolic phase was evaporated and purified by preparative HPLC (X-Terra RP-18, acetonitrile/water/aqueous NH₃ gradient from 10:90:0.2 to 95:5:0.2) to yield the title compound (33 mg, 52 %).

¹H-NMR (400 MHz, CDCl₃): δ 10.98 (s, 1H), 8.11 (d, J 2.1 Hz, 1H), 8.00 (d, J 2.2 Hz, 1H), 7.78 (d, J 9.2 Hz, 2H), 7.08 (d, J 8.9 Hz, 2H), 4.15 (t, J 6.4 Hz, 2H), 2.58 (t, J 7.3 Hz, 2H), 2.35 (s, 6H), 2.08 (quintet, J 7.0 Hz, 2H), 1.99 (septet, J 4.4 Hz, 1H), 1.00-0.94 (m, 2H), 0.56-0.52 (m, 2H).

APCI-MS m/z: 370.1 [MH⁺].

a) 5-Chloro-2-iodo-3-isopropenyl-1H-pyrrolo[2,3-b]pyridine

A mixture of 5-chloro-3-isopropenyl-2-(trimethylsilyl)-1H-pyrrolo[2,3-b]pyridine (0.56 g, 2.11 mmol), N-iodosuccinimide (0.71 g, 3.17 mmol) and dichloromethane (5 ml) was heated in a microwave reactor at 80° C. for 17 min. The reaction mixture was poured into aqueous Na₂S₂O₃ and extracted with dichloromethane. The combined organic layers were filtered through K₂CO₃ and silica gel to yield after evaporation the title compound (0.51 g, 79%).

APCI-MS m/z: 318.9 [MH⁺].

b) 5-Chloro-3-isopropenyl-2-(trimethylsilyl)-1H-pyrrolo[2,3-b]pyridine

A mixture of 5-chloro-3-iodopyridin-2-amine (1.00 g, 3.93 mmol), bis(triphenylphosphine)palladium(II) chloride (0.10 g, 0.14 mmol), 1,4-diazabicyclo(2,2,2)octane (1.00 g, 14.8 mmol), 2-methyl-4-trimethylsilyl-1-buten-3-yne (1.70 g, 12.3 mmol) and N,N-dimethylformamide (5 ml) was degassed and heated under argon atmosphere at 110° C. for 16 h. The reaction mixture was evaporated and the crude product was purified by column chromatography (silica gel, ethyl acetate-heptanes gradient from 0:100 to 100:0) to yield the subtitle compound (0.56 g, 54 %).

¹H-NMR (300 MHz, CDCl₃): δ 10.21 (s, 1H), 8.27 (d, J 2.3 Hz, 1H), 7.88 (d, J 2.3 Hz, 1H), 5.30 (sextet, J 1.4 Hz, 1H), 5.05 (q, J 1.1 Hz, 1H), 2.17 (s, 3H), 0.81 (s, 9H).

APCI-MS m/z: 265.0 [MH⁺].

### EXAMPLE 2

### {3-[4-(5-Chloro-3-cyclohex-1-en-1-yl-1H-pyrrolo[2,3-b]pyridin-2-yl)phenoxy]propyl}dimethylamine trifluoroacetate

The title compound (34 mg, 8 %) was synthesized from 5-chloro-3-cyclohex-1-en-1-yl-2-iodo-1H-pyrrolo[2,3-b]pyridine (0.35 g, 0.98 mmol) essentially as described in Example 1 and purified by preparative HPLC (RP-18, acetonitrile/water/TFA gradient from 10:90:0.1 to 95:5:0).

¹-NMR (400 MHz, CD₃OD): δ 8.10 (d, J 2.3 Hz, 1H), 7.82 (d, J 2.3 Hz, 1H), 7.67 (d, J 8.7 Hz, 2H), 7.06 (d, J 8.9 Hz, 2H), 5.88-5.85 (m, 1H), 4.19 (t, J 5.7 Hz, 2H), 3.39 (t, J 7.9 Hz, 2H), 2.97 (s, 6H), 2.30-2.22 (m, 4H), 2.13-2.08 (m, 2H), 1.77-1.71 (m, 4H).

APCI-MS m/z: 410.1 [MH³⁰ ].

### EXAMPLE 3

Tert-Butyl 3-(2-{4-[3-(dimethylamino)propoxy]phenyl}-5-methyl-1H-pyrrolo[2,3-b]pyridin-3-yl)piperidine-1-carboxylate

The title compound (8 mg, 17%) was synthesized from tert-butyl 3-(2-iodo-5-methyl-1H-pyrrolo[2,3-b]pyridin-3-yl)piperidine-1-carboxylate (90 mg, 0.20 mmol) essentially as described in Example 1. The compound was purified by preparative HPLC (RP-18, acetonitrile/water/AcOH gradient from 10:90:0.2 to 95:5:0.2).

¹H-NMR (300 MHz, CDCl₃): δ 11.45 (s, 1H), 8.00 (s, 1H), 7.90 (s, 1H), 7.43 (d, J 8.7 Hz, 2H), 7.06 (d, J 8.8 Hz, 2H), 4.29-4.18 (m, 1H), 4.06 (t, J 6.2 Hz, 2H), 3.24 (t, J 12.6 Hz, 1H), 3.07 (t, J 11.6 Hz, 1H), 2.88-2.75 (m, 4H), 2.52 (s, 6H), 2.44 (s, 3H), 2.24-2.03 (m, 3H), 1.95 (d, J 13.2 Hz, 1H), 1.77 (d, J 13.9 Hz, 1H), 1.64-1.51 (m, 1H), 1.47 (s, 9H).

APCI-MS m/z: 493.3 [MH⁺].

a) tert-Butyl 3-(2-iodo-5-methyl-1H-pyrrolo[2,3-b]pyridin-3-yl)piperidine-1-carboxylate

A mixture of rert-butyl 3-[5-methyl-2-(trimethylsilyl)-1H-pyrrolo[2,3-b]pyridin-3-yl]piperidine-1-carboxylate (0.12 g, 0.31 mmol), N-iodosuccinimide (89 mg, 0.40 mmol) and 1,2-dichloroethane (2 ml) was heated at 80° C. for 60 min. The mixture was poured into aqueous Na₂S₂O₃ and extracted with dichloromethane. The organic layers were filtered through K₂CO₃, evaporated and the crude product was purified by column the subtitle compound (89 mg, 65%).

APCI-MS m/z: 442.21 [MH⁺].

b) tert-Butyl 3-[5-methyl-2-(trimethylsilyl)-1H-pyrrolo[2,3-b]pyridin-3-yl]piperidine-1-carboxylate

A mixture of tert-butyl 3-[(2-trimethylsilyl)-ethynylpiperidine]-1-carboxylate (5.22 g, 18.5 mmol), bis(triphenylphosphine)palladium(II) chloride (0.72 g, 1.0 mmol), 1,4-diazabicyclo(2,2,2)octane (2.87 g. 25.6 mmol), 3-iodo-5-methylpyridin-2-amine (4.55 g, 19.4 mmol) and N,N-dimethylformamide (15 ml) was degassed and heated under an argon atmosphere at 110° C. for 19 h. The reaction mixture was evaporated and the crude product was purified by column chromatography (silica gel, ethyl acetate-heptanes gradient from 0:100 to 100:0) to yield the subtitle compound (4.24 g, 58%).

APCI-MS m/z: 388.3 [MH⁺].

c) tert-Butyl 3-[(2-trimethylsilyl)-ethynylperidine]-1-carboxylate

A solution of tert-butyl 3-ethynylpiperidine-1-carboxylate (5.42 g, 25.8 mmol) and dry tetrahydrofuran (30 ml) was cooled to −70° C. under an argon atmosphere and n-butyllithium solution in heptanes (21 ml, 33.6 mmol) was added. After 5 min, trimethylsilylchloride (4.3 ml, 34 mmol) was added. After an additional 15 min at −70° C. the volatiles were evaporated, the residue suspended in tert-butyl methyl ether and the slurry was filtered through a small plug of silica gel. The eluent was evaporated to yield the subtitle compound (6.71 g, 92%).

EIMS m/z: 281.3 [M⁺].

d) tert-Butyl 3ethynylpiperidine-1-carboxylate

To lithium diisopropylamine (2M in THF, 23.4 ml, 46.8 mmol) was added THF (50 ml) and the solution was cooled to −70° C. Trimethylsilyi-diazomethane (2M in heptane, 23.4 ml, 46.8 mmol) was added and after 30 min, tert-butyl 3-formylpipefidine-1-carboxylate (9.50 g, 44.5 mmol) in THF (5 ml) was added. After 30 min at −70° C. the mixture was allowed to slowly warm to room temperature during 150 min. The volatiles were evaporated and the crude product extracted with tert-butyl methyl ether from water. The organic layers were dried with Na₂SO₄, evaporated and purified by column chromatography (silica gel, ethyl acetate-heptanes gradient from 0:100 to 50:50) to yield the subtitle compound (5.92 g, 64%).

EIMS m/z: 209.2 [M⁺].

### EXAMPLE 4

### 2-(2-Furyl)-5-methyl-3-piperidin-3-y-1H-pyrrolo[2,3-b]pyridine

The title compound (72 mg, 46%) was synthesized from tert-butyl 3-(2-iodo-5-methyl-1H-pyrrolo[2,3-b]pyridin-3-yl)piperidine-1-carboxylate (Example 3a, 243 mg, 0.55 mmol) essentially as described in the synthesis of Example 3. but instead of using Dowex the mixture was treated with aqueous HCI and purified by preparative HPLC (RP-18, acetonitrile/water/aqueous NH₃ gradient from 10:90:0.2 to 95:5:0.2).

¹H-NMR (400 MHz, DMSO-d₆): δ 11.63 (s, 1H), 7.94 (s, 1H), 7.83 (d, J 1.5 Hz, 1H), 6.89 (d, J 3.4 Hz, 1H), 6.66 (q, J 1.7 Hz, 1H), 3.42-3.33 (m, 1H), 3.06-2.87 (m, 3H), 2.64 (t, J 12.2 Hz, 1H), 2.37 (s, 3H), 2.04 (q, J 12.3 Hz, 1H), 1.87-1.80 (m, 1H), 1.69 (d, J 12.9 Hz, 1H), 1.58-1.47 (m, 1H).

APCI-MS m/z: 382.2 [MH⁺].

### EXAMPLE 5

### 3-[2-(2-Furyl)-5-methyl-1H-pyrrolo[2,3-b]pyridin-3-yl]piperidine-1-carboxamide

A mixture of 2-(2-furyl)-5-methyl-3-piperidin-3-yl-1H-pyrrolo[2,3-b]pyridine (Example 4, 21 mg, 0.07 mmol), trimethylsilylisocyanate (3 drops) and EtOH (2 ml) was heated at 50° C. for 45 min. The volatiles were evaporated three times with EtOH to yield the title compound (17 mg, 70%).

¹H-NMR (400 MHz, CDCl₃) δ 10.30 (s, 1H), 8.97 (s, 1H), 7.94 (s, 1H), 7.70 (s, 1H), 7.61-7.54 (m, 2H), 6.85 (d, J 3.4 Hz, 1H), 6.58 (q, J 1.7 Hz, 1H), 4.19-4.11 (m, 1H), 4.03 (d, J 13.5 Hz, 1H), 3.48-3.39 (m, 1H), 2.97 (t, J 13.1 Hz, 1H), 2.48 (s, 3H), 2.19-2.06 (m, 4H).

APCI-MS m/z: 325.1 [MH⁺].

### EXAMPLE 6

### 5-Chloro-3-piperidin-4-yl-2-(1H-pyrrol-3-yl)-1H-pyrrolo[2,3-b]pyridine

The title compound (4 mg, 17%) was synthesized from tert-butyl 3-(5-chloro-2-iodo-1H-pyrrolo[2,3-b]pyridin-3-yl)pyrrolidine-1-carboxylate (synthesized by a similar procedure to that described in Example 3a, 35 mg, 0.08 mmol) essentially as described in the synthesis of Example 1, but instead of using Dowex the mixture was treated with aqueous HCl and purified by preparative HPLC (RP-18, acetonitrile/water/aqueous NH₃ gradient from 10:90:0.2 to 95:5:0.2).

APCI-MS m/z: 301.0 [MH⁺].

### EXAMPLE 7

### Tert-Butyl 4-(5-chloro-2-{4-[3-(dimethylamino)propoxy]phenyl}-1H-pyrrolo[2,3-b]pyridin-3-yl)piperidine-1-carboxylate

The title compound (16 mg, 18%) was synthesized from tert-butyl 3-(5-chloro-2-iodo-1H-pyrrolo[2,3-b]pyridin-3-yl)pyrrolidine-1-carboxylate (synthesized by a similar procedure to that described in Example 3a, 80 mg, 0.17 mmol) essentially as described in the synthesis of Example 1, and, without prior acidification, purified by HPLC (RP-18, acetonitrile/water/aqueous NH₃ gradient from 10:90:0.2 to 95:5:0.2).

APCI-MS m/z: 513.3 [MH⁺].

### EXAMPLE 8

### {3-[4-(5-Chloro-3-piperidin-4-yl-1H-pyrrolo[2,3-b]pyridin-2-yl)phenoxy]propyl}dimethylamine

The title compound (15 mg, 17%) was synthesized from tert-butyl 3-(5-chloro-2-iodo-1H-pyrrolo[2,3-b]pyridin-3-yl)pyrrolidine-1-carboxylate (96 mg, 0.21 mmol) essentially as described in Example 7.

¹H-NMR (300 MHz, CDCl₃): δ 10.90 (s, 1H), 8.18 (d, J 2.2 Hz, 1H), 8.04 (d, J 2.1 Hz, 1H), 7.47 (d, J 9.2 Hz, 2H), 7.08 (d, J 8.4 Hz, 2H), 4.13 (t, J 6.3 Hz, 2H), 3.24 (d, J 12.2 Hz, 1H), 3.11-2.98 (m, 1H), 2.73 (t, J 11.1 Hz, 2H), 2.57-2.43 (m, 2H), 2.29 (s, 6H), 2.21-1.93 (m, 6H), 1.81 (d, J 12.5 Hz, 1H)

APCI-MS m/z: 413.2 [MH⁺].

### EXAMPLE 9

### [3-(4-{5-Chloro-3-[1-(methylsulfonyl)piperidin-4-yl]-1H-pyrrolo[2,3-b]pyridin-2-yl}phenoxy)propyl]dimethylamine

Crude {3-[4-(5-chloro-3-piperidin-4yl-1H-pyrrolo[2,3-b]pyridin-2-yl)phenoxy]propyl}dimethylamine (Example 8, 50 mg, 0.12 mmol) and methanesulfonyl chloride (100 μl, 1.29 mmol) were dissolved in NMP (250 μl) and the pH was justed to 9 by additionof diisopropylethylamine. The reaction mixture was heated to 50° C. for 1 h, evaporated and purified by preparative HPLC (RP-18, acetonitrile/water/aqueous NH₃ gradient from 10:90:0.2 to 95:5:0.2) to give the title compound (6 mg, 10%).

¹H-NMR (300 MHz, CDCl₃): δ 10.23 (s, 1H), 8.09 (d, J 2.0 Hz, 1H), 8.02 (d, J 2.1 Hz, 1H), 7.44 (d, J 8.6 Hz, 2H), 7.06 (d, J 8.8 Hz, 2H), 4.18 (t, J 6.0 Hz, 2H), 3.96 (d, J 11.8 Hz, 2H), 2.93 (t, J 7.6 Hz, 2H), 2.83 (s, 3H), 2.73 (t, J 11.9 Hz, 2H), 2.62 (s, 6H), 2.38-2.18 (m, 5H), 1.93 (d, J 12.9 Hz, 2H).

APCI-MS m/z: 491.2 [MH⁺].

### EXAMPLE 10

### 4-(5-Chloro-2-{4-[3-(dimethylamino)propoxy]phenyl}-1H-pyrrolo[2,3-b]pyridin-3-yl)piperidine-1-carbaldehyde

The title compound (11 mg, 7%) was synthesized from {3-[4-(5chloro-3-piperidin-4-yl-1H-pyrrolo[2,3-b]pyridin-2-yl)phenoxy)propyl}dimethylamine (Example 8, 50 mg, 0.12 mmol), 1-hydroxybenzotriazole (60 mg, 0.45 mmol), EDC (49 mg, 0.26 mmol) and formic acid (60 μl) essentially as described in Example 9.

¹H-NMR (300 MHz, CDCl₃): δ 4.61 (d, J 13.3 Hz, 1H), 4.14 (t, J 6.2 Hz, 2H), 3.76 (d, J 13.0 Hz, 1H), 3.25-3.11 (m, 2H), 2.73-2.62 (m, 3H), 2.42 (s, 6H), 2.16-2.00 (m, 3H), 1.95-1.85 (m, 2H), 10.56 (s, 1H), 8.48 (s, 1H), 8.06 (d, J 2.3 Hz, 1H), 7.93 (d, J 2.0 Hz, 1H), 7.46 (d, J 8.8 Hz, 2H), 7.08 (d, J 8.4 Hz, 2H).

APCI-MS m/z: 441.2 [MH⁺].

### EXAMPLE 11

### 4-(5-Chloro-2-{4-[3-(dimethylamino)propoxy]phenyl}-1H-pyrrolo[2,3-b]pyridin-3-yl)piperidine-1-carboxamide

The title compound (10 mg, 18%) was synthesized from {3-[4-(5-chloro-3-piperidin-4-yl-1H-pyrrolo[2,3-b]pyridin-2-yl)phenoxy]propyl}dimethylamine (Example 8, 50 mg, 0.12 mmol) and trimethylsilylisocyanate (50 mg, 0.51 mmol) essentially as described in Example 9, but before purification the mixture was acidified with aqueous HCl.

¹H-NMR (300 MHz, CDCl₃): δ 10.46 (s, 1H), 8.09-8.05 (m, 1H), 7.97 (d, J 2.2 Hz, 2H), 7.45 (d, J 8.7 Hz, 2H), 7.08 (d, J 8.8 Hz, 2H), 4.60 (s, 1H), 4.15-4.07 (m, 3H), 3.16-2.82 (m, 2H), 2.51 (t, J 7.3 Hz, 2H), 2.29 (s, 6H), 2.22-1.96 (m, 3H), 1.90-1.61 (m, 2H), 1.39-1.16 (m, 2H).

APCI-MS m/z: 456.2 [MH⁺].

### EXAMPLE 12

### 3-(2-{4-[3-(Dimethylamino)propoxy]phenyl}-5-methyl-1H-pyrrolo[2,3-b]pyridin-3-yl)-N,N-dimethylpiperidine-1-carboxamide

The title compound (1 mg, 4%) was synthesized from N,N-dimethyl-3-[4-(5-methyl-3-piperidin-3-yl-1H-pyrrolo[2,3-b]pyridin-2-yl)phenoxy]propan-1-amine (20 mg, 0.05 mmol) and dimethylcarbonyl chloride (80 μl) essentially as described in Example 9.

¹H-NMR (400 MHz, DMSO-d₆): δ 11.50 (s, 1H), 8.01 (s, 1H), 7.99 (s, 1H), 7.47 (d, J 8.8 Hz, 2H), 7.04 (d, J 8.7 Hz, 2H), 4.06 (t, J 6.3 Hz, 2H), 3.63-3.51 (m, 2H), 3.30-3.22 (m, 1H), 3.06-2.86 (m, 2H), 2.71 (s, 6H), 2.39 (s, 3H), 2.18 (s, 6H), 2.16-2.05 (m, 1H), 1.93-1.78 (m, 3H), 1.70 (d, J 14.5 Hz, 1H), 1.55-1.45 (m, 1H).

APCI-MS m/z: 464.3 [MH⁺].

a) N,N-Dimethyl-3-[4-(5-methyl-3-piperidin-3-yl-1H-pyrrolo[2,3-b]pyridin-2-yl)phenoxy]propan-1-amine

A mixture of tert-butyl 3-(2-{4-[3-(dimethylamino)propoxy]phenyl}-5-methyl-1H-pyrrolo[2,3-b]pyridin-3-yl)piperidine-1-carboxylate (Example 3, 1.60 g, 3.25 mmol), 1,4-dioxane (30 ml) and aqueous concentrated HCl (7 ml) was stirred at room temperature for 15 min. The solvents were evaporated off and the residue was dissolved in and evaporated twice from EtOH. Purification by preparative HPLC (RP-18, acetonitrile/water/acetic acid gradient from 10:90:0.2 to 95:5:0.2) yielded the title compound (12 mg, 16%).

APCI-MS m/z: 393.2 [MH⁺].

### EXAMPLE 13

### 3-(2-{4-[3-(Dimethylamino)propoxy]phenyl}-5-methyl-1H-pyrrolo[2,3-b]pyridin-3-yl)-N-isopropy]piperidine-1-carboxamide

The title compound (2 mg, 8%) was synthesized from N,N-dimethyl-3-[4-(5-methyl-3-piperidin-3-yl -1H-pyrrolo[2,3-b]pyridin-2-yl)phenoxy]propan-1-amine (Example 12a, 20 mg, 0.05 mmol) and isopropylisocyanate (80 μl) essentially as described in Example 9.

¹H-NMR (400 MHz, DMSO-d₆): δ 11.49 (s, 1H), 8.01 (s, 1H), 7.99 (s, 1H), 7.49 (d, J 8.7 Hz, 2H), 7.03 (d, J 8.8 Hz, 2H), 6.12 (d, J 7.7 Hz, 1H), 4.06 (t, J 6.3 Hz, 2H), 4.03-3.97 (m, 1H), 3.75 (sextet, J 6.6 Hz, 1H), 3.21 (t, J 13.1 Hz, 1H), 2.97-2.76 (m, 2H), 2.39 (s, 3H), 2.16 (s, 6H), 2.14-2.01 (m, 1H), 1.92-1.76 (m, 2H), 1.67 (d, J 14.3 Hz, 1H), 1.39 (t, J 14.5 Hz, 1H), 1.29-1.22 (m, 2H), 1.02 (q, J 3.0 Hz, 6H).

APCI-MS m/z: 478.5 [MH⁺].

### EXAMPLE 14

### Dimethyl[3-(4-{5-methyl-3-[1-(pyrrolidin-1-ylcarbonyl)piperidin-3-yl]-1H-pyrrolo[2,3-b]pyridin-2-yl}phenoxy)propyl]amine

The title compound (2 mg, 8%) was synthesized from N,N-dimethyl-3-[4-(5-methyl-3-piperidin-3-yl-1H-pyrrolo[2,3-b]pyridin-2-yl)phenoxy]propan-1-amine (Example 12a, 20 mg, 0.05 mmol) and pyrrolidinecarbonyl chloride (80 μl) essentially as described in Example 9.

¹H-NMR (400 MHz, DMSO-d₆): δ 11.50 (s, 1H), 8.02-7.98 (m, 2H), 7.47 (d, J 8.8 Hz, 2H), 7.04 (d, J 8.7 Hz, 2H), 4.06 (t, J 6.4 Hz, 2H), 3.71 (d, J 13.0 Hz, 2H), 3.63 (d, J 13.1 Hz, 2H), 3.24-3.18 (m, 4H), 3.07-2.86 (m, 2H), 2.52 (s, 3H), 2.38-2.31 (m, 2H), 2.15 (s, 6H), 1.91-1.81 (m, 3H), 1.75-1.67 (m, 5H), 1.54-1.42 (m, 1H).

APCI-MS m/z: 490.5 [MH⁺].

### EXAMPLE 15

### [3-(4-{3-[1-(Isopropylsulfonyl)piperidin-3-yl]-5-methyl-1H-pyrrolo[2,3-b]pyridin-2-yl}phenoxy)propyl]dimethylamine

The title compound (2 mg, 8%) was synthesized from N,N-dimethyl-3-[4-(5-methyl-3-piperidin-3-yl-1H-pyrrolo[2,3-b]pyridin-2-yl)phenoxy]propan-1-amine (Example 12a, 20 mg, 0.05 mmol) and isopropylsulfonyl chloride (80 μl) essentially as described in the synthesis of Example 9.

¹H-NMR (400 MHz, DMSO-d₆): δ 11.56 (s, 1H), 8.06-8.00 (m, 2H), 7.46 (d, J 8.9 Hz, 2H), 7.05 (d, J 8.7 Hz, 2H), 4.08 (t, J 6.4 Hz, 2H), 3.66 (q, J 13.1 Hz, 2H), 3.42 (t, J 12.1 Hz, 1H), 3.13 (t, J 12.5 Hz, 1H), 3.05-2.96 (m, 2H), 2.39 (s, 1H), 2.36-2.09 (m, 8H), 1.98-1.88 (m, 3H), 1.81 (t, J 15.0 Hz, 2H), 1.55-1.43 (m, 2H), 1.25-1.18 (m, 7H).

APCI-MS m/z: 499.5 [MH⁺].

### EXAMPLE 16

### (3-{4-[3-(1-acetylpiperidin-3-yl)-5-methyl-1H-pyrrolo[2,3-b]pyridin-2-yl]phenoxy}propyl)dimethylamine

The title compound (2 mg, 9%) was synthesized from N,N-dimethyl-3-[4-(5-methyl-3-piperidin-3-yl-1H-pyrrolo[2,3-b]pyridin-2-yl)phenoxy]propan-1-amine (Example 12a, 20 mg, 0.05 mmol) and acetic anhydride (80 μl) essentially as described in Example 9.

¹H-NMR (400 MHz, DMSO-d₆): δ 11.58-11.47 (m, 1H), 8.05-7.99 (m, 2H), 7.49-7.41 (m, 2H), 7.11-7.00 (m, 2H), 4.43 (q, J 17.2 Hz, 1H), 4.06 (t, J 13.7 Hz, 2H), 3.84 (t, J 26.1 Hz, 1H), 3.60 (t, J 16.8 Hz, 1H), 3.27-3.20 (m, 1H), 3.04 (t, J 18.3 Hz, 1H), 2.98-2.78 (m, 1H), 2.73-2.64 (m, 1H), 2.44-2.30 (m, 5H), 2.16 (s, 6H), 2.02 (s, 1.7H), 1.93 (s, 1.3H), 1.91-1.68 (m, 4H).

APCI-MS m/z: 435.4 [MH⁺].

### EXAMPLE 17

### 3-(2-{4-[3-(Dimethylamino)propoxy]phenyl}-5-methyl-1H-pyrrolo[2,3-b]pyridin-3-yl)-N-methylperidine-1-carbothioamide

The title compound (3 mg, 13%) was synthesized from N,N-dimethyl-3-[4-(5-methyl-3-piperidin-3-yl-1H-pyrrolo[2,3-b]pyridin-2-yl)phenoxy]propan-1-amine (Example 12a, 20 mg, 0.05 mmol) and methylisothiocyanate (100 mg) essentially as described in Example 9.

¹H-NMR (400 MHz, DMSO-d₆): δ 11.45 (s, 1H), 7.99 (s, 1H), 7.90 (s, 1H), 7.43 (d, J 8.7 Hz, 2H), 7.06 (d, J 8.8 Hz, 2H), 4.06 (t, J 6.2 Hz, 2H), 3.09-2.99 (m, 1H), 2.93 (s, 3H), 2.90-2.80 (m, 2H), 2.77 (s, 3H), 2.47-2.31 (m, 5H), 2.16 (s, 6H), 2.09 (t, J 11.3 Hz, 1H), 1.87 (quintet, J 6.9 Hz, 1H), 1.73 (t, J 14.3 Hz, 3H).

APCI-MS m/z: 466.4 [MH⁺].

### EXAMPLE 18

### 2-(2-{[3-(2-[4-[3-(Dimethylamino)propoxy]phenyl}-5-methyl-1H-pyrrolo[2,3-b]pyridin-3-yl)piperidin-1-yl]sulfonyl]ethyl)-1H-isoindole-1,3(2H)-dione

The title compound (2 mg, 6%) was synthesized from N,N-dimethyl-3-[4-(5-methyl-3-piperidin-3-yl-1H-pyrrolo[2,3-b]pyridin-2-yl)phenoxy]propan-1-amine (Example 12a, 20 mg, 0.05 mmol) and 2-(1,3-dioxo-1,3-dihydro-isoindol-2-yl)ethanesulfonyl chloride (100 mg) essentially as described in Example 9.

¹H-NMR (400 MHz, DMSO-d₆): δ 11.56 (s, 1H), 8.03-7.99 (m, 2H), 7.90-7.83 (m, 4H), 7.43 (d, J 8.6 Hz, 2H), 7.05 (d, J 8.7 Hz, 2H), 4.05 (t, J 6.3 Hz, 2H), 3.98-3.92 (m, 2H), 3.66 (d, J 12.0 Hz, 1H), 3.57 (d, J 13.8 Hz, 1H), 3.44 (t, J 7.1 Hz, 2H), 3.12-2.99 (m, 3H), 2.39 (s, 3H), 2.39 (d, J 13.8 Hz, 1H), 3.44 (t, J 7.1 Hz, 2H), 3.12-2.99 (m, 3H), 2.39 (s, 3H), 2.39 (s, 6H), 2.13-2.04 (m, 1H), 1.92-1.79 (m, 5H), 1.60-1.46 (m, 2H).

APCI-MS m/z: 630.5 [MH⁺].

### EXAMPLE 19

### 3-[3-(2-{4-[3-(Dimethylamino)propoxy]phenyl}-5-methyl-1H-pyrrolo[2,3-b]pyridin-3-yl)piperidin-1-yl)piperidin-1-yl]pyrrolidine-2,5-dione

The title compound (2 mg, 8%) was synthesized from N,N-dimethyl-3-[4-(5-methyl-3-piperidin-3-yl-1H-pyrrolo[2,3-b]pyridin-2-yl)phenoxy]propan-1-amine (Example 12a, 20 mg, 0.05 mmol) and maleimide (100 mg) essentially as described in Example 9.

¹H-NMR (400 MHz, DMSO-d₆): δ 11.47 (s, 1H), 11.15 (s, 0.3H), 8.00 (s, 1H), 7.89 (s, 1H), 7.46-7.40 (m, 2H), 7.10-7.03 (m, 2H), 4.07 (t, J 6.6 Hz, 2H), 3.90-3.84 (m, 1H), 3.16-2.99 (m, 2H), 2.87-2.64 (m, 4H), 2.39 (s, 3H), 2.18 (s, 6H), 1.94-1.84 (m, 4H), 1.79-1.66 (m, 3H), 1.58-1.43 (m, 2H).

APCI-MS m/z: 490.4 [MH⁺].

### EXAMPLE 20

### Dimethyl[3-(4-{5-methyl-3-[1-(methylsulfonyl)piperidin-3-yl]-1H-pyrrolo[2,3-b]pyridin-2-yl}phenoxy)propyl]amine

The title compound (1 mg, 4%) was synthesized from N,N-dimethyl-3-[4-(5-methyl-3-piperidin-3-yl-1H-pyrrolo[2,3-b]pyridin-2-yl)phenoxy]propan-1-amine (Example 12a, 20 mg, 0.05 mmol) and methylsulfonyl chloride (80 μl) essentially as described in Example 9.

APCI-MS m/z: 471.4 [MH⁺].

### EXAMPLE 21

### 5-Bromo-2-(4-methoxy-phenyl)-3-piperazin-1-yl-1H-pyrrolo[2,3-b]pyridine trifluoroacetate

4-[5-Bromo-2-(4-methoxy-phenyl)-1H-pyrrolo[2,3-b]pyridin-3-yl]-piperazine-1-carboxylic acid tert-butyl ester bis TFA salt (6 mg, 0.0084 mmol) was dissolved in dichloromethane (5 ml) and TFA (1 ml) was added. The mixture was heated to reflux for 30 minutes and then concentrated in vacuo. The residue was recrystallized from ethyl acetate to give the pure title compound as a mono-TFA salt, white powder (2 mg, 48%).

¹H-NMR (acetone-d₆): δ 12.00 (1H, s); 8.58 (2H, bs); 8.24 (2H, s); 8.01 (2H, d); 7.04 (2H, d); 3.81 (3H, s); 3.35-3.20 (8H, m).

APCI-MS m/z: 387.0 [MH⁺].

### EXAMPLE 22

### 5-Bromo-2-(4-methoxpyhenyl)-3-(4-methylpiperazin-1-yl)-1H-pyrrolo[2,3-b]pyridine

2-Bromo-1-(4-methoxyphenyl)-ethanone (1.14 g, 5 mmol)) was dissolved in N,N-dimethylformamide (20 ml). 1-Methylpiperazine (1.04 g, 10 mmol) was added and after 10 minutes the reaction mixture was diluted with water (200 ml) and the mixture extracted with ethyl acetate (3×200 ml). The combined organic phase was washed with brine (2×20 ml) and dried (MgSO₄) and the solvents evaporated. This afforded the crude piperazinomethylketone as a pale yellow oil that solidified upon standing (540 mg, 43%) which was used in the next step without further purification. This ketone (248 mg, 1 mmol) and (5-bromo-pyridin-2-yl)-hydrazine (188 mg, 1 mmol) were heated together at 230° C. for 1 h. When cool, the dark brown glassy solid was dissolved in N,N-dimethylformamide (2 ml) and subjected to preparative HPLC. This afforded a crude product (27 mg, 7%) that was approximately 90% pure. This maiterial was purified by preparative HPLC (RP-18, acetonitrile/water/trifluoroacetic acid gradient from 10:90:0.1 to 95:5:0.1) to give pure (>99%) product as a white powder (5 mg).

¹H-NMR (DMSO-d₆): δ 12.01 (1H, s); 9.60 (1H, bs); 8.24 (2H, m); 8.02 (2H, d); 7.03 (2H, d); 3.81 (3H, s); 3.58-3.45 (4H, m); 3.55-3.20 (4H, m); 2.91 (3H, s).

APCI-MS m/z: 401.0 [MH⁺].

### EXAMPLE 23

### 4-[5-Bromo-2-(4-methoxy-phenyl)-1H-pyrrolo[2,3-b]pyridin-3-yl]-piperazine-1-carboxylic acid tert-butyl ester bis-trifluoroacetate

2-Bromo-1-(4-methoxyphenyl)-ethanone (1.14 g, 5 mmol) was dissolved in N,N-dimethylformamide (20 ml). 1-Piperazine-1-carboxylic acid tert-butyl ester (931 mg, 5 mmol) and DIEA (0.85 ml, 5 mmol) were added and after 10 minutes the reaction mixture was diluted with water (200 ml) and the mixture extracted with ethyl acetate (3×200 ml). The combined organic phase was washed with brine (2×20 ml) and dried (MgSO₄) and the solvents evaporated. This afforded the crude protected piperazinomethylketone as a pale yellow oil that solidified upon standing (1.65 g, 99%) which was used in the next step without further purification. This ketone (334 mg, 1 mmol) and (5-bromo-pyridin-2-yl)-hydrazine (188 mg, 1 mmol) were heated together for 30 minutes at 110° C. and then at 200° C. for 45 minutes. The crude product was purified twice by preparative HPLC (RP-18, acetonitrile/water/trifluoroacetic acid gradient from 10:90:0.1 to 95:5:0.1) to give the pure (>99%) product as a white powder (12 mg, 2.5%).

¹H-NMR (acetone-d₆): δ 10.78 (1H, s); 8.24(1H, s); 8.16 (1H, s); 8.10 (2H, d); 7.03 (2H, d); 3.84 (3H, s); 3.57 (4H, t); 3.18 (4H, t); 2.93 (1.8H, bs); 1.46 (9H, s).

APCI-MS m/z: 487.2 [MH⁺].

### EXAMPLE 24

### 5-Bromo2-(4-methoxyphenyl)-3-morpholin-4-yl-1H-pyrrolo[2,3-b]pyridine

2-Bromo-1-(4-methoxyphenyl)-ethanone (5 g, 25 mmol) was dissolved in N,N-dimethylformamide (15 ml). Morpholine (4.35 g, 50 mmol) was added and the solution turned yellow and became warm. As it was allowed to cool, morpholine hydrobromide crystallized out and was removed by filtration. The filtrate was diluted with tolueue (100 ml) and (5-bromo-pyrdin-2-yl)-hydrazine (4.7 g, 25 mmol) was added. The resulting mixture was refluxed for 14 h, while azeotropically removing water. The solvents were removed in vacuo and the resulting red-brownish oil was purified by column chromatography (silica gel, ethyl acetate/heptane gradient 0:100 to 100:0). The second eluting component was collected and concentrated in vacuo to give the hydrazone as a brown oil (6.8 g, 72%). This oil (1.07 g, 2.8 mmol) was heated to 200-205° C. for 40 minutes and then allowed to cool. The dark brown glassy product was dissolved in boiling acetonitrile and the title compound crystallised as this solution was allowed to cool. The product was collected by filtration and thoroughly washed with acetonitrile. This crude product was further recrystallized from acetone/dichloromethane to afford a pale yellow powder (8 mg, 0.80%).

¹H-NMR (DMSO-d₆): δ 11.99 (1H, bs); 8.35 (1H, s); 8.24 (1H, s); 8.12 (2H, d); 7.48 (2H, t); 7.35 (1H, t); 3.73 (4H, t); 3.13 (4H, t).

APCI-MS m/z: 358.2 [MH⁺].

### EXAMPLE 25

### 5-Bromo-3-(4-methanesulfonylpiperazin-1-yl)-2-(4-methoxy-phenyl)-1H-pyrrolo[2,3-b]pyridine

Piperazine-1-carboxylic acid tert-butyl ester (1.86 g, 10 mmol) was dissolved in pyridine (15 ml) and methanesulfonyl chloride (1.14 g, 10 mmol) was added. The mixture turned yellow and become warm. After 10 minutes, the reaction was diluted with water (150 ml) and left standing, whereupon the precipitate was collected. To this precipitate, dichloromethane (15 ml) and trifluoroacetic acid (2.5 ml) were added. The mixture was heated to boiling and then left to cool. The solvent was removed and the resulting yellow oil was dissolved in ethyl acetate (20 ml). Crystals of 1-(methylsulfonyl)piperazine trifluoroacetate were collected by filtration (0.871 g, 72%). 1-(Methylsulfonyl)piperazine trifluoroacetate was dissolved in N,N-dimethylformamide (5 ml) and N-ethyl-N,N-diisopropylamine (2.2 ml, 13 mmol) was added, followed by 2-bromo-1-(4-methoxyphenyl)-ethanone (0.72 g, 3.1 mmol). After 5 minutes, the reaction mixture was poured into water (50 ml) and crystals of 2-(4-methanesulfonyl-piperazin-1-yl)-1-(4-methoxy-phenyl) -ethanone were collected (1.11 g, 67%). Part of this ketone (312 mg, 1 mmol) and (5-bromopyridin-2-yl)-hydrazine (188 mg, 1 mmnol) were fused together at 210° C. for 30 minutes. After cooling, the crude product was crystallized from acetonitrile to give the title compound (31 mg, 7%).

¹H-NMR (DMSO-d₆): δ 11.92 (1H, s); 8.31 (1H, s); 8.20 (1H, s); 8.04 (2H, d); 7.05 (2H, d); 3.81 (3H, s); 3.7-3.3 (8H, m); 2.97 (3H, s).

APCI-MS m/z: 465.4 [MH⁺].

### EXAMPLE 26

### 4-[5-Bromo-2-(4-methoxy-phenyl)-1H-pyrrolo[2,3-b]pyridin-3-yl]-piperazine-1-carbaldehyde

2-Bromo-1-(4-methoxyphenyl)-ethanone (2.24 g, 10 mmol) was dissolved in N,N-dimethylformamide (20 ml). Piperazine-1-carbaldehyde (2.3 g, 20 mmol) and N-ethyl-N,N-diisopropylamine (0.85 ml; 5 mmol) were added and after 30 minutes the reaction mixture was diluted with ethyl acetate (100 ml) and the mixture washed with brine (4×100 ml). The organic phase was dried (MgSO₄) and the solvent evaporated off. The residue was dissolved in a mixture of toluene (50 ml) and (5-bromo-pyridin-2-yl)-hydrazine (1.88 g, 10 mmol). The mixture was heated at reflux for 4 h and then allowed to cool. The solvent was removed in vacuo and the residue chromatographed (silica gel, ethyl acetate/heptane 1:1). Crude 4-[2-[(5-bromopyridin-2-yl)hydrazono]-2-(4-methoxyphenyl)ethyl]piperazine-1-carbaldehyde was heated at 210° C. for 20 minutes. The crude product was purified by preparative HPLC (RP-18, acetonitrile/water/trifluoroacetic acid gradient from 10:90:0.1 to 95:5:0.1). Appropriate fractions were evaporated and the solid was washed with a 1:1 acetonitrile/water mixture to give the title compound (12 mg, 0.3%).

¹H-NMR (DMSO-d₆): δ 11.90 (1H, s); 8.32 (1H, s); 8.19 (1H, s); 8.09 (2H, d); 8.08 (1H, s); 7.05 (2H, d); 3.80 (3H, s); 3.54 (2H, t); 3.50 (2H, t); 3.15 (2H, t); 3.06 (3H, t).

¹³C-NMR (DMSO-d₆): δ 160.8; 158.8; 144.8; 142.0; 131.8; 128.8; 128.7; 123.3; 122.2; 118.7; 113.8; 110.2; 55.1; 52.4; 51.4; 45.7; 40.1.

APCI-MS m/z: 415.3 [MH⁺].

### Screen

Itk LANCE TRF Assay

The Itk kinase assay utilized recombinant human Itk kinase domain fused with GST (Glutathione S-Transferase). The protein was expressed in High five insect cells, purified in one step on an affinity chromatography glutathione column and stored in 50 mM Tris/HCl (pH 7.6), 150 mM NaCl, 5% (w/v) mannitol, 1 mM DTT, 30% glycerol at −70° C. The kinase substrate used in the assay was a biotinylated peptide derived from the Src-optimal substrate (Nair et at, J. Med. Chem., 38: 4276, 1995; biotin-AEEEIYGEFEAKKKK).

The assay additions were as follows: Test compounds (or controls; 1 μL in 100% DMSO) were added to black 96-well flat-bottomed plates (Greiner 655076) followed by 20 μL Itk in assay buffer and the reaction was started by adding 20 μL ATP and peptide substrate in assay buffer. The assay buffer constitution during phosphorylation was: 50 mM HEPES (pH 6.8), 10 mM MgCl₂, 0.015% Brij 35, 1 mM DTT, 10% glycerol, 160 ng/well Itk, 2 μM peptide substrate and 50 μM ATP. The assay was stopped after 50 minutes (RT) by adding 150 μL ice-cold Stop solution (50 mM Tris/HCl, pH 7.5, 10 mM EDTA, 0.9% NaCl and 0.1% BSA) together with LANCE reagents (2 nM PT66-Eu³⁺, Wallac AD0069 and 5 μg/ml Streptavidin-APC, Wallac AD0059. Both concentrations were final in stopped assay solution). The plates were measured on a Wallac 1420 Victor 2 instrument with TRF settings after 1 h incubation, and the ratio (665 signal/615 signal)*10000 was used to calculate the inhibition values. IC₅₀ values were determined using XLfit.

When tested in the above screens, the compounds of Examples 1 to 26 gave IC₅₀ values for inhibition of Itk activity of less than 25 μM, indicating that the compounds of the invention are expected to possess useful therapeutic properties.

Representative results are shown in the following Table:

| | Inhibition of Kinase Itk |
|---|---|
| Compound | (IC₅₀ μM) |
| Example 5 | 0.26 |
| Example 8 | 0.18 |
| Example 21 | 0.09 |

1. A compound of formula (I): 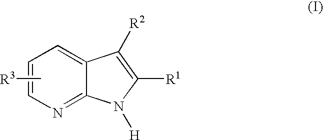
wherein:
R¹ represents phenyl or a five or six membered aromatic heterocyclic ring containing 1 to 3 heteroatoms selected independently from O, S and N; said phenyl or aromatic heterocyclic ring being optionally substituted by one or more substituents selected independently from halogen, C1 to 4 alkyl, C1 to 4 alkoxy, CO₂R⁴ or a group -K-L-M;
K represents O, NR¹² or a bond;
L represents C1 to 4 alkyl optionally further substituted by OH or OMe; or L represents a bond;
M represents NR¹³R¹⁴ or OR¹⁵;
R¹³ and R¹⁴ independently represent H or C1 to 4 alkyl; or the group —NR¹³R¹⁴ together represents a saturated 5 to 7 membered azacyclic ring optionally incorporating one further heteroatom selected from O, S and NR¹⁶;
R¹⁶ represents H, C1 to 4 alkyl or C2 to 4 alkanoyl;
R² represents a saturated or partially unsaturated 3 to 7 membered ring, optionally including 1 or 2 heteroatoms independently selected from O, N and S(O)ₙ and optionally incorporating 1 or 2 carbonyl groups; and optionally substituted by halogen, OH, C1 to 4 alkyl, C1 to 4 alkoxy, CHO, C2 to 4 alkanoyl, C1 to 4 alkylsulphonyl, CO₂R⁵, C(Z)NR¹⁷R¹⁸ or pyrrolidine-2,5-dione; said C1 to 4 alkylsulphonyl group being optionally further substituted by 1H-isoindole-1,3(2H)-dione;
Z represents O or S;
R¹⁷ and R¹⁸ independently represent H or C1 to 4 alkyl; or the group —NR¹⁷R¹⁸ together represents a saturated 5 to 7 membered azacyclic ring optionally incorporating one further heteroatom selected from O, S and NR¹⁹;
R³ represents H, halogen, C1 to 4 alkyl, C1 to 4 alkoxy or cyano;
R⁴, R⁵, R¹², R¹⁵ and R¹⁹ independently represent H or C1 to 4 alkyl;
n represents an integer 0, 1 or 2;
and pharmaceutically acceptable salts thereof.

2. A compound according to claim 1 wherein R³ represents halogen or methyl.

3. A compound according to claim 1 wherein K represents O.

4. A compound of formula (I), according to claim 1, which is:
{3-[4-(5-chloro-3-cyclopropyl-1H-pyrrolo[2,3-b]pyridin-2-yl)phenoxy]propyl}dimethylamine;
{3-[4-(5-chloro-3-cyclohex-1-en-1-yl-1H-pyrrolo[2,3-b]pyridin-2-yl)phenoxy]propyl}dimethylamine;
tert-butyl 3-(2-{4-[3-(dimethylamino)propoxy]phenyl}-5-methyl-1H-pyrrolo[2,3-b]pyridin-3-yl)piperidine-1-carboxylate;
2-(2-furyl)-5-methyl-3-piperidin-3-yl-1H-pyrrolo[2,3-b]pyridine;
3-[2-(2-furyl)-5-methyl-1H-pyrrolo[2,3-b]pyridin-3-yl]piperidine-1-carboxamide;
5-chloro-3-piperidin-4-yl-2-(1H-pyrrol-3-yl)-1H-pyrrolo[2,3-b]pyridine;
tert-butyl 4-(5-chloro-2-{4-[3-(dimethylamino)propoxy]phenyl}-1H-pyrrolo[2,3-b]pyridin-3-yl)piperidine-1-carboxylate;
{3-[4-(5-chloro-3-piperidin-4-yl-1H-pyrrolo[2,3-b]pyridin-2-yl)phenoxy]propyl}dimethylamine;
[3-(4-{5-chloro-3-[1-(methylsulfonyl)piperidin-4-yl]-1H-pyrrolo[2,3-b]pyridin-2-yl}phenoxy)propyl]dimethylamine;
4-(5-chloro-2-{4-[3-(dimethylamino)propoxy]phenyl}-1H-pyrrolo[2,3-b]pyridin-3-yl)piperidine-1-carbaldehyde;
4-(5-chloro-2-{4-[3-(dimethylamino)propoxy]phenyl}-1H-pyrrolo[2,3-b]pyridin-3-yl)piperidine-1-carboxamide;
3-(2-{4-[3-(dimethylamino)propoxy]phenyl}-5-methyl-1H-pyrrolo[2,3-b]pyridin-3-yl)-N,N-dimethylpiperidine-1-carboxamide;
3-(2-{4-[3-(dimethylamino)propoxy]phenyl}-5-methyl-1H-pyrrolo[2,3-b]pyridin-3-yl)-N-isopropylpiperidine-1-carboxamide;
dimethyl[3-(4-{5-methyl-3-[1-(pyrrolidin-1-ylcarbonyl)piperidin-3-yl]-1H-pyrrolo[2,3-b]pyridin-2-yl}phenoxy)propyl]amine;
[3-(4-{3-[1-(isopropylsulfonyl)piperidin-3-yl]-5-methyl-1H-pyrrolo[2,3-b]pyridin-2-yl}phenoxy)propyl]dimethylamine;
(3-{4-[3-(1-acetylpiperidin-3-yl)-5-methyl-1H-pyrrolo[2,3-b]pyridin-2-yl]phenoxy}propyl)dimethylamine;
3-(2-{4-[3-(dimethylamino)propoxy]phenyl}-5-methyl-1H-pyrrolo[2,3-b]pyridin-3-yl)-N-methylpiperidine-1-carbothioamide;
2-(2-{[3-(2-{4-[3-(dimethylamino)propoxy]phenyl}-5-methyl-1H-pyrrolo[2,3-b]pyridin-3-yl)piperidin-1-yl]sulfonyl}ethyl)-1H-isoindole-1,3(2H)-dione;
3-[3-(2-{4-[3-(dimethylamino)propoxy]phenyl}-5-methyl-1H-pyrrolo[2,3-b]pyridin-3-yl)piperidin-1-yl]pyrrolidine-2,5-dione;
dimethyl[3-(4-{5-methyl-3-[1-(methylsulfonyl)piperidin-3-yl]-1H-pyrrolo[2,3-b]pyridin-2-yl}phenoxy)propyl]amine;
5-bromo-2-(4-methoxy-phenyl)-3-piperazin-1-yl-1H-pyrrolo[2,3-b]pyridine;
5-bromo-2-(4-methoxyphenyl)-3-(4-methylpiperazin-1-yl)-1H-pyrrolo[2,3-b]pyridine;
4-[5-bromo-2-(4-methoxy-phenyl)-1H-pyrrolo[2,3-b]pyridin-3-yl]-piperazine-1-carboxylic acid tert-butyl ester;
5-bromo-2-phenyl-3-morpholin-4-yl-1H-pyrrolo[2,3-b]pyridine;
5-bromo-3-(4-methanesulfonylpiperazin-1-yl)-2-(4-methoxy-phenyl)-1H-pyrrolo[2,3-b]pyridine;
4-[5-bromo-2-(4-methoxy-phenyl)-1H-pyrrolo[2,3-b]pyridin-3-yl]-piperazine-1-carbaldehyde;
or a pharmaceutically acceptable salt of any one thereof.

5. (canceled)

6. A pharmaceutical formulation comprising a compound of formula (I), as defined in claim 1, or a pharmaceutically acceptable salt thereof, optionally in admixture with a pharmaceutically acceptable diluent or carrier.

7. A method of treating, or reducing the risk of, a human disease or condition in which inhibition of Itk kinase activity is beneficial which comprises administering to a person suffering from or susceptible to such a disease or condition, a therapeutically effective amount of a compound of formula (I), as defined in claim 1, or a pharmaceutically acceptable salt thereof.

8. (canceled)

9. The method according to claim 7 wherein the disease is asthma.

10. The method according to claim 7 wherein the disease is allergic rhinitis.

11. A process for the preparation of a compound of formula (I), as defined in claim 1, and optical isomers and racemates thereof and pharmaceutically acceptable salts thereof, which comprises:
a) reaction of a compound of formula (II): 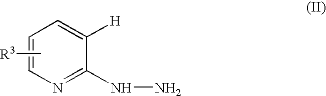
in which R³ is as defined in claim 1, with a compound of formula (III): 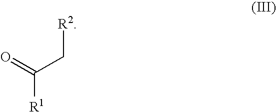
in which R¹ and R² are as defined in claim 1; or
b) arylation of a compound of formula (IV) 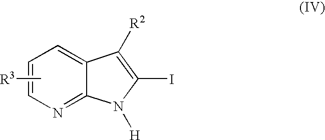
wherein R² and R³ are as defined in claim 1, with a boronic acid of formula R¹—B(OH)₂ wherein R¹ is as defined in claim 1;
and where desired or necessary converting the resultant compound of formula (I), or another salt thereof, into a pharmaceutically acceptable salt thereof, or converting one compound of formula (I) into another compound of formula (I); and where desired converting the resultant compound of formula (I) into an optical isomer thereof.
